(19) United States
(12) Patent Application Publication
(10) Pub. No.: US 2018/0312629 A1
(43) Pub. Date: Nov. 1, 2018

(54) NOVEL BRANCHED SULFUR-CONTAINING POLYMERS

(71) Applicants: PIVERT, Venette (FR); UNIVERSITÉ DE BORDEAUX, Bordeaux (FR); IINSTITUT DES CORPS GRAS ETUDES ET RECHERCHES TECHNIQUES - ITERG, Pessac (FR); INSTITUT POLYTECHNIQUE DE BORDEAUX, Talence (FR); CENTRE NATIONAL DE LA RECHERCHE SCIENTIFIQUE, Paris (FR)

(72) Inventors: Blandine TESTUD, Talence (FR); Etienne GRAU, Talence (FR); Daniel TATON, Camarsac (FR); Henri CRAMAIL, Sainte Terre (FR); Didier PINTORI, Talence (FR)

(73) Assignees: PIVERT, Venette (FR); UNIVERSITÉ DE BORDEAUX, Bordeaux (FR); INSTITUT DES CORPS GRAS ETUDES ET RECHERCHES TECHN IQUES - ITERG, Pessac (FR); INSTITUT POLYTECHNIQUE DE BORDEAUX, Talence (FR); CENTRE NATIONAL DE LA RECHERCHE SCIENTIFIQUE, Paris (FR)

(21) Appl. No.: 15/770,307

(22) PCT Filed: Oct. 28, 2016
(86) PCT No.: PCT/EP2016/076170

§ 371 Date: Apr. 23, 2018

(30) Foreign Application Priority Data
Oct. 29, 2015 (FR) 15 60389

(51) Int. Cl. C08G 63/688; C07C 323/52

(52) U.S. Cl. CPC C08G 63/6886; C08G 63/85; C07C 323/52

(57) ABSTRACT

The present invention relates to a compound that may be obtained according to a method comprising at least one polymerization step of at least one monomer of formula (I) below:

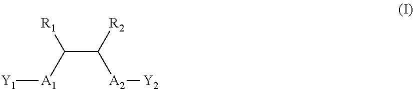

wherein the polymerization step is carried out in the presence of a catalyst selected from the group consisting of: Zn(OAc)₂, Ti(OBu)₄, Ti(OiPr)₄, Sb₂O₃, stannous octanoate, dibutyltin oxide, 7-methyl-1,5,7-triazabicyclo[4.4.0]dec-5-ene, NaOMe, 1,5,7-triazabicyclo[4.4.0]dec-5-ene and Lipase B Candida Antartica.

The present invention relates to novel branched or hyper-branched polymers obtained from sulfur monomers. The invention also relates to a method for preparing branched or hyperbranched polymers as mentioned above, as well as the monomers used to prepare them.

In recent decades, branched (and/or hyperbranched) polymers have gained increasing importance because of their particular architecture. These macromolecules are characterized by a branched structure of variable density and a large number of functional groups.

Because of their unique properties, branched polymers have a very wide range of applications. They may be used as additives, hardeners for thermosetting polymers, crosslinking agents or adhesives, dispersing agents, compatibilizing agents or rheology modifiers.

At present, there is a need for branched polymers, and, more particularly, for branched and biobased polymers.

The present invention aims to provide novel branched polymers, and, more particularly, biosourced branched polymers, having satisfactory rheological and thermomechanical properties.

The present invention also aims to provide a method for preparing branched polymers that is simple to implement, efficient and green.

The present invention also aims to provide a method for preparing branched polymers, carried out in the absence of solvent.

Thus, the present invention relates to a compound obtainable by a method comprising at least one polymerization step of at least one monomer of formula (I) below:

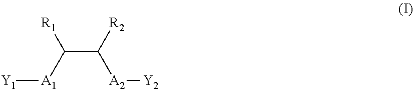

in which:
Y₁ is H, an OH group or a COORₐ group, wherein Rₐ represents H or a linear or branched alkyl group comprising from 1 to 6 carbon atoms,
Y₂ is an OH or COORₐ group, wherein Rₐ is as defined above, it being understood that:
   when Y₁ is H, then Y₂ is COORₐ,
   when Y₁ is OH, then Y₂ is OH,
   when Y₁ is COORₐ, then Y₂ is COORₐ,
wherein R₁ and R₂ are defined as follows:
   either R₁ is H and R₂ is a group of formula —S-A₃-X,
   or R₁ is a group of formula —S-A₃-X and R₂ is H,
      wherein A₃ represents a linear or branched alkylene radical comprising from 1 to 12, preferably from 1 to 6 carbon atoms, and
      wherein X represents an OH or COOR_{b} group, wherein R_{b} is H or a linear or branched alkyl group comprising from 1 to 6 carbon atoms,
A₁ is a linear or branched alkylene radical comprising from 1 to 20, in particular from 2 to 20, carbon atoms, optionally substituted by at least one OH group, wherein the alkylene radical is further substituted by a side chain —S-A₃-X, wherein A₃ and X are as defined above,
A₂ is a linear or branched alkylene radical comprising from 1 to 20, in particular from 2 to 20, carbon atoms, optionally substituted by at least one OH group, wherein it is understood that at least one of the groups X, Y₁ and Y₂ is COORₐ or COOR_{b}, and wherein at least one of the groups A₁, A₂, X, Y₁ or Y₂ comprises an OH group, wherein the total number of COORₐ, COOR_{b} and OH functions is at least 3,
wherein the polymerization step is carried out in the presence of a catalyst selected from the group consisting of: Zn(OAc)₂, Ti(OBu)₄, Ti(OiPr)₄, Sb₂O₃, stannous octanoate, dibutyltin oxide, 7-methyl-1,5,7-triazabicyclo[4.4.0]dec-5-ene, NaOMe, 1,5,7-triazabicyclo[4.4.0]dec-5-ene and Lipase B Candida Antartica.

The compounds according to the invention are polymers obtained exclusively from type AB₂ or A₂B synthons. These compounds are also designated as branched or even hyper-branched polymers, or polyesters of branched architecture or branched polyesters.

These compounds therefore comprise at least one dendritic unit, which comprises several branches.

The polymeric compounds according to the invention are obtained by polymerization of a monomer of formula (I) in the presence of a catalyst as defined above.

According to the invention, the term “polymer” denotes a compound obtained by polymerization according to the aforementioned method and comprising at least two repeating units derived from the monomers of formula (I).

The monomers used according to the invention are monomers comprising at least one hydroxyl function (via Y₁, Y₂ or X or also via the radicals A₁ or A₂ when they comprise a lateral substituent OH) and at least one an acid or ester function COORₐ or COOR_{b} (via Y₁, Y₂ or X). The presence of at least one hydroxyl function and at least one ester or acid function thus makes it possible to form ester functions and thus to obtain polyesters. In addition, the monomers of formula (I) must comprise at least a total of 3 reactive functions selected from the hydroxyl and acid or ester functions in order to obtain the aforementioned AB₂ or A₂B synthons. In particular, the monomers according to the invention may comprise a hydroxyl function and two ester or acid functions or they may comprise two hydroxyl functions and an ester or acid function.

According to one embodiment, the monomers according to the invention comprise two hydroxyl functions and an ester function or two ester functions and a hydroxyl function.

According to one embodiment, when Y₁ is H and Y₂ is COORₐ, then R₁ or R₂ is a —S-A₃-OH group. According to another embodiment, when Y₁ is H and Y₂ is COORₐ, and A₁ and/or A₂ comprises a lateral OH substituent, then R₁ or R₂ may be a —S-A₃-COOR_{b} group or a —S-A₃-OH group.

According to one embodiment, when Y₁═Y₂═OH, then R₁ or R₂ is a —S-A₃-COOR_{b} group.

According to one embodiment, when Y₁═Y₂═COORₐ, then R₁ or R₂ is a —S-A₃-OH group.

The monomers used according to the invention are monomers comprising at least one sulfur group, i.e. at least one group of the formula —S-A₃-X, wherein X and A₃ are as defined above.

According to one embodiment, the monomers used according to the invention comprise several identical or different groups of formula —S-A₃-X, wherein X and A₃ are as defined above.

According to one embodiment, the monomers used according to the invention comprise at least one group of formula —S-A₃-OH, wherein A3 is as defined above. According to this embodiment, these monomers therefore comprise at least one particularly reactive primary alcohol function.

In the context of the present invention, the term “alkyl” designates a linear or branched saturated aliphatic hydrocarbon group comprising, unless stated otherwise, from 1 to 12, in particular from 1 to 6 carbon atoms, and preferably from 1 to 4 carbon atoms. By way of example of alkyl groups, mention may be made of methyl, ethyl, propyl, butyl, pentyl or hexyl groups.

In the context of the present invention, the term “alkylene” (or “alkylidene”) denotes a linear or branched divalent radical comprising, unless stated otherwise, from 1 to 20 carbon atoms, and preferably from 2 to 10 carbon atoms.

According to one embodiment, in the formula (I) as defined above, A₁ may be an alkylene radical as defined above and further comprising a lateral hydroxyl substituent. According to this embodiment, the monomer of formula (I) therefore comprises a secondary alcohol function.

According to one embodiment, in the formula (I) as defined above, A₁ may be an alkylene radical as defined above and further comprising a side chain —S-A₃-X, wherein A₃ and X are as defined above.

According to one embodiment, in the formula (I) as defined above, A₂ may be an alkylene radical as defined above and further comprising a lateral hydroxyl substituent. According to this embodiment, the monomer of formula (I) therefore comprises a secondary alcohol function.

In the formula (I) as defined above, the radicals A₁ and A₂ are thus connected to each other by a —CH(R₁)—CH(R₂)— group which corresponds to either a group of formula (A) or a group of formula (B) as defined below:

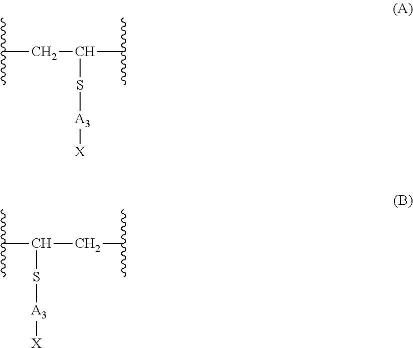

wherein A₃ and X are as defined in formula (I).

According to one embodiment, the compound according to the invention as defined above is obtained by polymerization of a monomer of formula (II) below:

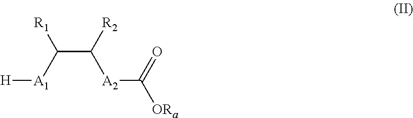

wherein A₁, R₁, R₂, A₂ and Rₐ are as defined in formula (I).

The compounds of formula (II) correspond to the compounds of formula (I) in which Y₁═H and Y₂═COORₐ.

According to this embodiment, X is OH when A₁ and A₂ are alkylene radicals unsubstituted by OH.

According to this embodiment, when at least one of the radicals A₁ and A₂ is an alkylene radical substituted by OH, then X may be —OH or —COOR_{b}.

Among the monomers of formula (II), mention may be made of the monomers of formula (II-a) below:

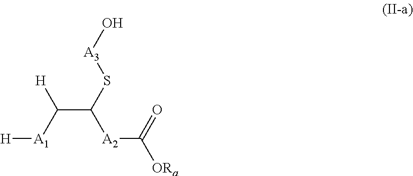

in which A₁, A₃, A₂ and Rₐ are as defined above.

The compounds of formula (II-a) correspond to the compounds of formula (I) in which Y₁═H, Y₂═COORₐ, R₁═H and R₂═-S-A₃-OH.

Among the monomers of formula (II), mention may also be made of the monomers of formula (II-b) below:

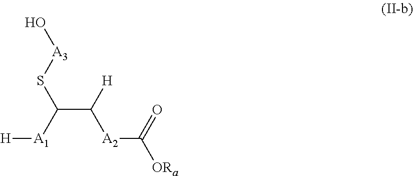

In which A₁, A₃, A₂ and Rₐ are as defined above.

The compounds of formula (II-b) correspond to the compounds of formula (I) in which Y₁═H, Y₂═COORₐ, R₁═-S-A₃-OH and R₂═H.

Among the monomers used according to the invention, mention may also be made of the monomers of formula (II-1) below:

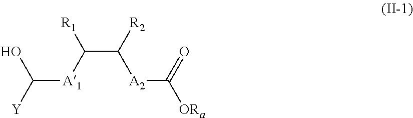

in which:
R₁, R₂, A₂ and Rₐ are as defined in formula (I),
A′₁ is a linear or branched alkylene radical comprising from 1 to 6 carbon atoms, and
Y is a linear or branched alkyl group comprising from 1 to 10 carbon atoms.

The compounds of formula (II-1) correspond to the compounds of formula (I) in which Y₁=-A′₁-CH(OH)—Y and Y₂═COORₐ.

According to this embodiment, the formula (II-1) includes a hydroxyl function and a COORₐ function, while X may be indifferently OH or COOR_{b}.

Among the monomers of formula (II-1), mention may be made of the monomers of formula (II-1-a) below:

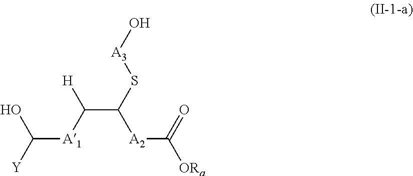

in which Y, A′₁, A₃, A₂ and Rₐ are as defined above.

The compounds of formula (II-1-a) correspond to the compounds of formula (I) in which Y₁=-A′₁-CH(OH)—Y, Y₂═COORₐ, R₁═H and R₂═-S-A₃-OH.

Among the monomers of formula (II-1), mention may be made of the monomers of formula (II-1-b) below:

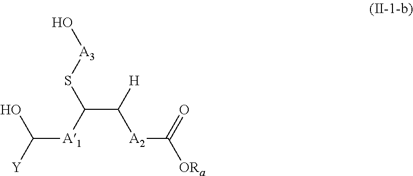

in which Y, A′₁, A₃, A₂ and Rₐ are as defined above.

The compounds of formula (II-1-b) correspond to the compounds of formula (I) in which Y₁=-A′₁-CH(OH)—Y, Y₂═COORₐ, R₁═-S-A₃-OH and R₂═H.

Among the monomers of formula (II-1), mention may be made of the monomers of formula (II-1-c) below:

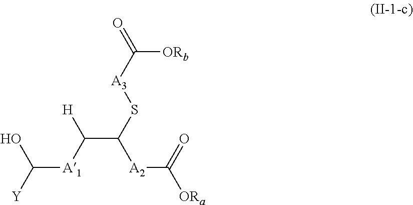

in which Y, A′₁, A₃, R_{b}, A₂ and Rₐ are as defined above.

The compounds of formula (II-1-c) correspond to the compounds of formula (I) in which Y₁=-A′₁-CH(OH)—Y, Y₂═COORₐ, R₁═H and R₂═-S-A₃-COOR_{b}.

Among the monomers of formula (II-1), mention may be made of the monomers of formula (II-1-d) below:

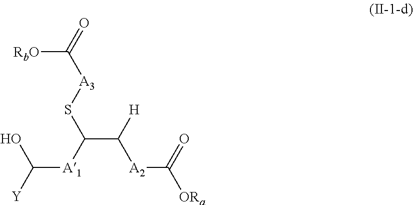

in which Y, A′₁, A₃, R_{b}, A₂ and Rₐ are as defined above.

The compounds of formula (II-1-d) correspond to the compounds of formula (I) in which Y₁=-A′₁-CH(OH)—Y, Y₂═COORₐ, R₁═S-A₃-COOR_{b} and R₂═H.

According to one embodiment, X is OH in formulas (I), (II) and (II-1) as defined above.

According to one embodiment, in formulas (I), (II) and (II-1) as defined above, X is COOR_{b}, while R_{b} is as defined in formula (I).

Among the monomers used according to the invention, mention may also be made of the monomers of formula (III) below:

in which:

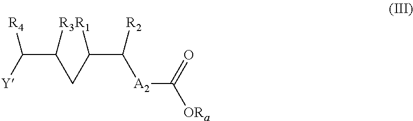

A₂ and Rₐ are as defined in formula (I),
R₁ and R₂ are defined as follows:
   either R₁ is H and R₂ is a group of formula —S-A₃-OH,
   or R₁ is a group of the formula —S-A₃-OH and R₂ is H, while A₃ is as defined in formula (I),
R₃ and R₄ are defined as follows:
   either R₃ is H and R₄ is a group of formula —S-A₃-OH,
   or R3 is a group of the formula —S-A₃-OH and R₄ is H, while A₃ is as defined in formula (I),
Y′ is a linear or branched alkyl group comprising from 1 to 10 carbon atoms.

The compounds of formula (III) correspond to compounds of formula (I) in which Y₁═-CH₂—CH(R₃)—CH(R₄)—Y′ and Y₂═COORₐ.

Among the monomers used according to the invention, mention may also be made of the monomers of formula (IV) below:

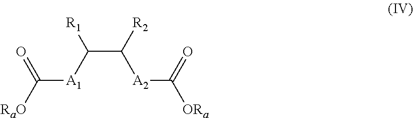

in which:
A₁, A₂ and Rₐ are as defined in formula (I), and
A₁, A₂ and Rₐ are defined as follows:
   either R₁ is H and R₂ is a group of formula —S-A₃-OH,
   or R₁ is a group of the formula —S-A₃-OH and R₂ is H, while A₃ is as defined in formula (I).

The compounds of formula (IV) correspond to the compounds of formula (I) in which Y₁═Y₂═COORₐ.

Among the monomers used according to the invention, mention may also be made of the monomers of formula (V) below:

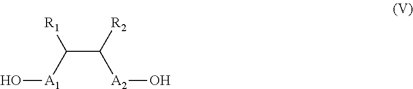

in which:
A₁ and A₂ are as defined in formula (I), and
R₁ and R₂ are defined as follows:
   either R₁ is H and R₂ is a group of formula —S-A₃-COOR_{b},
   or R₁ is a group of formula —S-A₃-COOR_{b} and R₂ is H,
   while A₃ and R_{b} are as defined in formula (I).

The compounds of formula (V) correspond to the compounds of formula (I) in which Y₁═Y₂═OH.

According to a preferred embodiment, in the abovementioned formulas of monomers according to the invention, A₁ comprises from 6 to 12, preferably from 8 to 10, carbon atoms. Preferably, A₁ is a linear alkylene radical. In particular, A₁ may be a linear alkylene radical comprising 8 or 9 carbon atoms.

According to a preferred embodiment, in the abovementioned formulas of monomers according to the invention, A₂ comprises from 6 to 12, preferably from 8 to 10, carbon atoms. Preferably, A₂ is a linear alkylene radical. In particular, A₂ may be a linear alkylene radical comprising 7, 8 or 9 carbon atoms.

According to a preferred embodiment, in the abovementioned formulas of monomers according to the invention, A₃ comprises from 1 to 4 carbon atoms. Preferably, A₃ is a linear alkylene radical. In particular, A₃ may be a linear alkylene radical having 1 or 2 carbon atoms.

According to a preferred embodiment, in the abovementioned formulas of monomers according to the invention, Rₐ comprises from 1 to 4 carbon atoms. Preferably, Rₐ is a linear alkyl group. In particular, Rₐ is a methyl group.

According to a preferred embodiment, in the abovementioned formulas of monomers according to the invention, R_{b} comprises from 1 to 4 carbon atoms. Preferably, R_{b} is a linear alkyl group. In particular, R_{b} is a methyl group.

Among the preferred monomers used according to the invention, mention may be made of the following monomers:

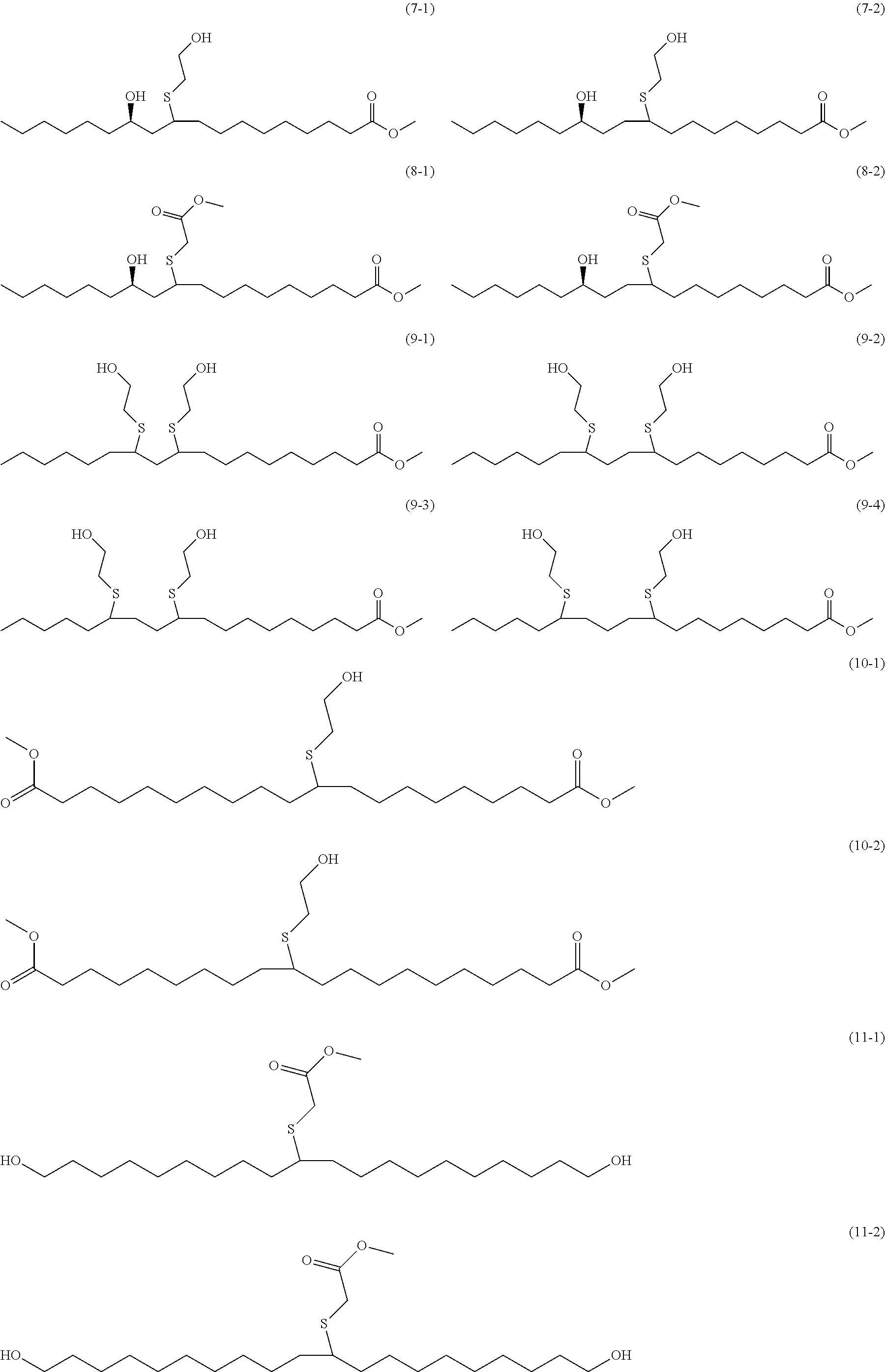

The present invention also relates to a method for preparing a compound as defined above, namely a polymer comprising at least one polymerization step of a monomer of formula (I) as defined above, in the presence a catalyst selected from the group consisting of: Zn(OAc)₂, Ti(OBu)₄, Ti(OiPr)₄, Sb₂O₃, stannous octanoate, dibutyltin oxide, 7-methyl-1,5,7-triazabicyclo[4.4.0]dec-5-ene, NaOMe, 1,5,7-triazabicyclo [4.4.0]dec-5-ene and Lipase B Candida Antartica.

Preferably, the catalyst used in the method according to the invention is Zn(OAc)₂, 1,5,7-triazabicyclo[4.4.0]dec-5-ene or NaOMe (sodium methanolate).

The method according to the invention therefore consists in polymerizing the monomer of formula (I) to obtain a polyester, wherein this is effected in the presence of a catalyst.

According to one embodiment, in the method according to the invention, the catalyst content ranges from 0.05% to 20%, preferably from 0.05% to 10% by weight, relative to the total weight of monomer of formula (I).

According to a preferred embodiment, the aforementioned polymerization step is carried out (a) by heating the monomer of formula (I) as defined above in the presence of the abovementioned catalyst, at a temperature T₁ of 3° C. to 130° C. for a period of 1 hour to 48 hours, for example under a stream of nitrogen, then, optionally, (b) under dynamic vacuum at the temperature T₁ for a period of 1 hour to 48 hours.

Preferably, this heating step (a) is carried out at a temperature ranging from 90° C. to 120° C.

Thus, according to one embodiment, the polymerization method according to the invention is carried out by bringing the monomer and the catalyst into contact at a temperature T₁ and then by placing them under vacuum.

The method according to the invention may further comprise an additional optional step, namely a step (c) of heating to a temperature T₂ of 90° C. to 180° C., for a period of 1 hour to 48 hours.

The method according to the invention has the advantage of not using a solvent.

In addition, this method makes it possible to obtain, a satisfactory yield of polymers having interesting thermomechanical properties.

The present invention also relates to the monomers of formula (I) as defined above, as well as the monomers of formulas (II), (II-a), (II-b), (II-1), (II-1-a), (II-1-b), (II-1-c), (II-1-d), (III), (IV) and (V) as defined above.

It also relates to the monomers of formulas (7-1), (7-2), (8-1), (8-2), (9-1), (9-2), (9-3), (9-4), (10-1), (10-2), (11-1) and (11-2) as defined above.

Throughout this application, the wording “comprising a” or “having a” is understood to mean “comprising at least one” or “having at least one” unless the contrary is specified.

Throughout the above description, unless stated otherwise, the term “comprised between x and y” corresponds to an inclusive range, i.e. the x and y values are included in the range.

### EXAMPLES

Ricinoleate (99%) and methyl oleate (99%) were supplied by NuChek Prep.

The compounds 1,5,7-triazabicyclo[4.4.0]dec-5-ene (98%), zinc acetate (99.99%), dibutyltin oxide (98%), 7-methyl-1,5,7-triazabicyclo[4.4.0]dec-5-ene, sodium methanolate, 2,2-dimethoxy-2-phenylacetophenone, 2nd generation Grubbs catalyst and stannous octanoate (95%), were purchased from Sigma Aldrich. Ti (OBu)₄ and Ti (OiPr)₄ were provided by Acros Organics, Sb₂O₃ and 10-undecen-1-ol by Alfa Aesar.

Finally, methyl undecenoate (98%) was purchased from TCI Europe. Lipase B Candida Antartica (Immozyme), immobilized on acrylic resin was provided by Chiral Vision. All the reagents were used without further purification.

### Example 1: Preparation of the Monomers (7)

The monomers (7) have the following general formula:

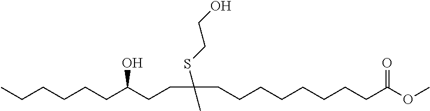

They therefore correspond to the aforementioned monomers (7-1) and (7-2).

The monomers (7) were prepared from ricinoleic acid methyl ester by grafting 2-mercaptoethanol. The thiol was introduced in excess of the double bond (3 equivalents), and no solvent was added. These additions were catalyzed under UV irradiation by the 2,2-dimethoxy-2-phenylacetophenone (DMPA).

This preparation method may be represented by the following reaction scheme:

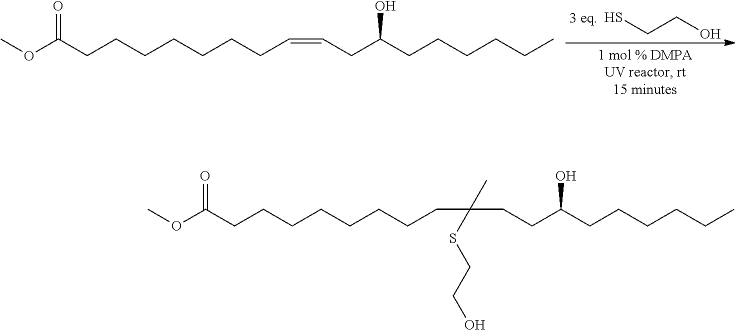

In a 50 ml flask equipped with a magnetic bar, are mixed 10 g of methyl ricinoleate (32 mmol) and 7.5 g of 2-mercaptoethanol (96 mmol). 82 mg of DMPA (0.32 mmol) are then added. The reaction medium is then placed under UV irradiation in a dedicated reactor for this purpose (400 W, 315 nm≤λ≤400 nm). The progress of the reaction is monitored by 1H NMR spectroscopy until complete disappearance of the characteristic signals of the double bond. 15 minutes are needed to achieve total conversion. Excess 2-mercaptoethanol is then removed from the medium by aqueous washings. The products are finally purified by flash chromatography on a silica column, on the basis of a cyclohexane/ethyl acetate elution (85/15:v/v). The monomers (7) are obtained in the form of colorless viscous liquids of good purity (96.8% determined by gas chromatography). The yields achieved are 92%.

### Example 2: Preparation of the Monomers (8)

The monomers (8) have the following general formula:

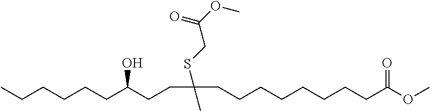

They therefore correspond to the aforementioned monomers (8-1) and (8-2).

The monomers (8) were prepared according to a protocol similar to that of Example 1, starting from the methyl ester of ricinoleic acid by grafting methyl thioglycolate. The thiol was introduced in excess of the double bond (3 equivalents), and no solvent was added. These additions were catalyzed under UV irradiation with 2,2-dimethoxy-2-phenylacetophenone (DMPA).

This preparation method may be represented by the following reaction scheme:

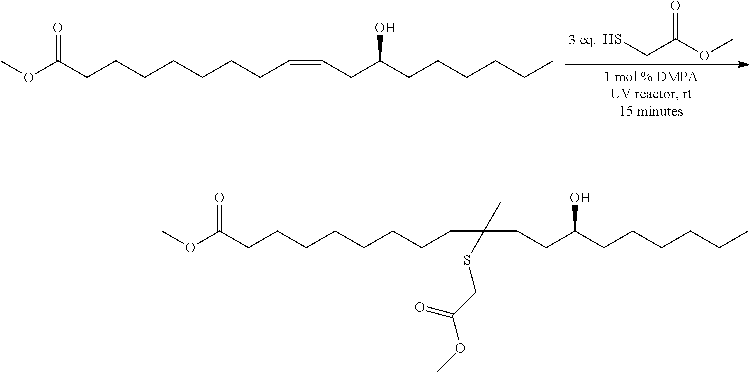

In a 50 ml flask equipped with a magnetic bar, are mixed 5 g of methyl ricinoleate (16 mmol) and 5 g of methyl thiolgycolate (47 mmol). 41 mg of DMPA (0.16 mmol) are then added thereto. The reaction medium is then placed under UV irradiation in a dedicated reactor for this purpose (400 W, 315 nm≤λ≤400 nm). The progress of the reaction is monitored by 1H NMR spectroscopy until complete disappearance of the characteristic signals of the double bond. It takes 10 minutes to achieve total conversion. Excess methyl thioglycolate is then removed by vacuum distillation. The products are finally purified by flash chromatography on a silica column, on the basis of a cyclohexane/ethyl acetate elution (90/10:v/v). The monomers (8) are obtained in the form of colorless viscous liquids of good purity (94% determined by gas chromatography). The yields achieved are 90%.

### Example 3: Preparation of the Monomers (9)

The monomers (9) have the following general formula:

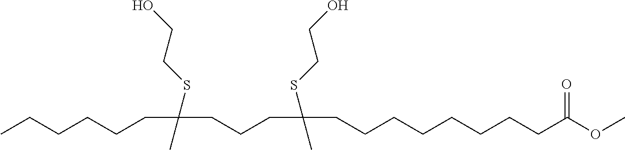

They therefore correspond to the aforementioned monomers (9-1), (9-2), (9-3) and (9-4).

The monomers (9) were prepared according to the method described in Example 1 for the monomers (7) but from the methyl linoleate (C18:2) in the absence of DMPA. The 2-mercaptoethanol was introduced in excess (3 equivalents relative to the unsaturation). No solvent was added, as the medium was completely homogeneous.

This preparation method may be represented by the following reaction scheme:

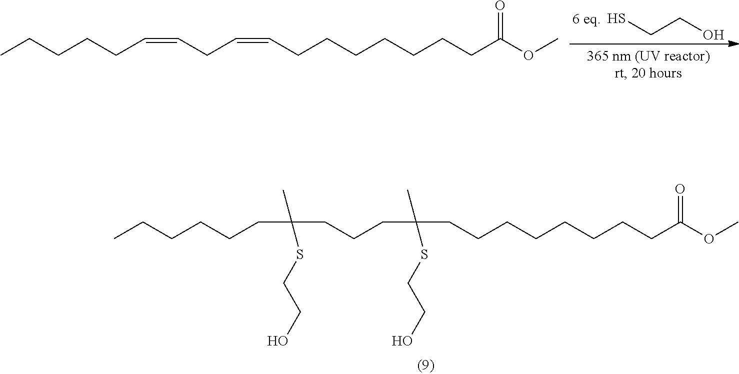

In a 50 ml flask fitted with a magnetic bar, are mixed 5 g of methyl linoleate (17 mmol) and 8 g of 2-mercaptoethanol (102 mmol). The reaction medium is then placed under UV irradiation in a dedicated reactor for this purpose (400 W, 315 nm≤λ≤400 nm). The progress of the reaction is monitored by 1H NMR spectroscopy until complete disappearance of the characteristic signals of the double bond. 20 hours are needed to achieve total conversion. Excess 2-mercaptoethanol is then removed from the medium by aqueous washings. The monomers (9) are obtained in the form of colorless viscous liquids. The yields achieved are 96%.

### Example 4: Preparation of the Monomers (10)

The monomers (10) have the following general formula:

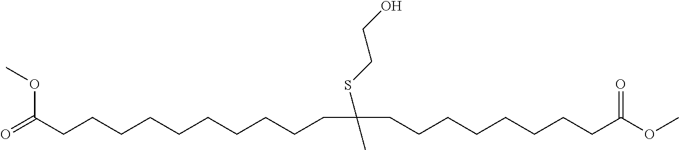

They therefore correspond to the aforementioned monomers (10-1) and (10-2).

These monomers are prepared from the methyl decenoate, wherein a C11 fatty acid derivative has a terminal double bond. The reaction scheme below involves two steps:

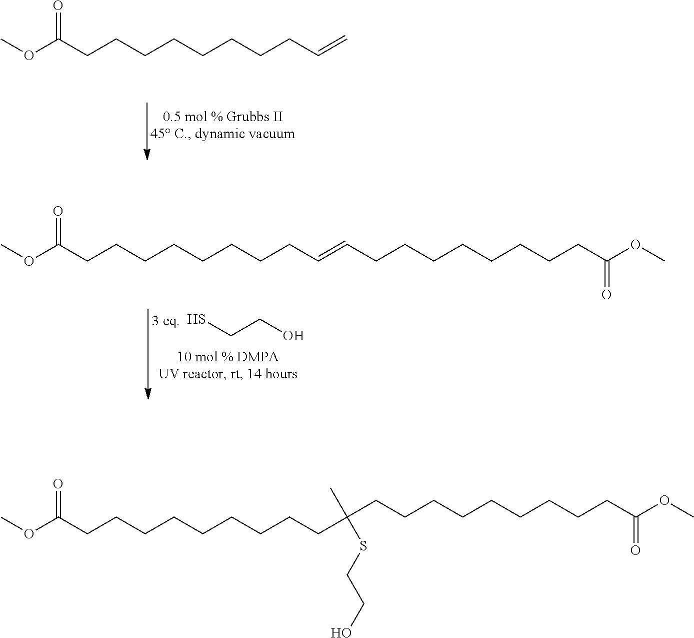

At first, a diester is generated by metathesis of the fatty substance. The alcohol-antagonist function is then introduced by reaction of “thiol-ene” with 2-mercaptoethanol.

The monomers (10) are then formed by “thiol-ene” reaction with 2-mercaptoethanol. These additions were conducted in a UV reactor (A=365 nm) and were catalyzed by DMPA and were not optimized. The monomers (10) are thus obtained in the form of more or less colored viscous liquids.

In a 50 ml Schlenk flask previously dried, evacuated and equipped with a magnetic bar, are introduced 15 g of methyl undecenoate (76 mmol) and 0.3 g of 2nd generation Grubbs catalyst (0.38 mmol). The reaction medium is then heated to 45° C. and stirred under dynamic vacuum for 40 hours. Once the reaction medium has returned to ambient temperature, 2 ml of ethylvinyl ether are added in order to deactivate the Grubbs catalyst. The excess ethylvinyl ether is then removed by vacuum distillation. The product is then purified by chromatography on a silica column. The eluent used is a mixture composed of 95% cyclohexane and 5% ethyl acetate. The metathesized diester is obtained in the form of a white solid. The yield of the step is 66%.

The monomers (10) are then formed by reaction of thiol-ene with 2-mercaptoethanol. To do this, 5 g of the intermediate diester (14 mmol) and 3.2 g of 2-mercaptoethanol (41 mmol) are dissolved in 10 ml of dichloromethane. 0.35 g of DMPA (1.4 mmol) are added thereto. The reaction medium is then placed under UV irradiation in a dedicated reactor for this purpose (400 W, 315 nm≤λ≤400 nm). The progress of the reaction is monitored by 1H NMR spectroscopy until complete disappearance of the characteristic signals of the double bond. Excess 2-mercaptoethanol is then removed from the medium by aqueous washings. The products are finally purified by flash chromatography on a silica column, on the basis of a cyclohexane/ethyl acetate elution (90/10:v/v). The yield of the step is 95%.

### Example 5: Preparation of the Monomers (11)

The monomers (11) have the following general formula:

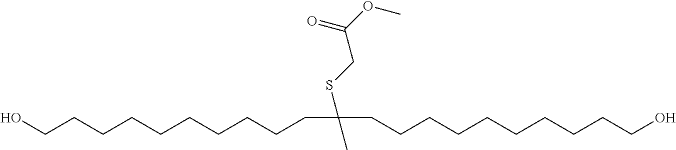

They therefore correspond to the aforementioned monomers (11-1) and (11-2).

These monomers are prepared from the undecen-1-ol, a C11 fatty acid derivative having a terminal double bond. The reaction scheme below involves two steps:

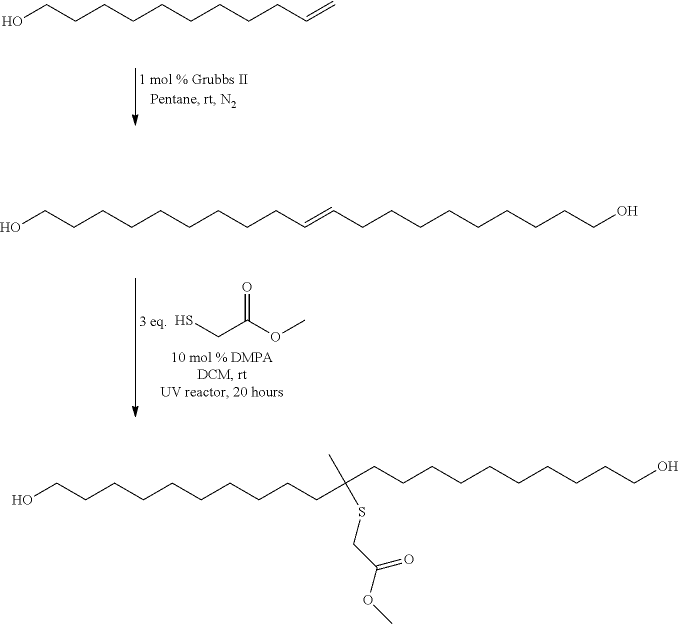

At first, a diol is generated by metathesis of the fatty substance. The ester-antagonist function is then introduced by reaction of “thiol-ene” with methyl thioglycolate.

The monomers (11) are then formed by “thiol-ene” reaction with methyl thioglycolate. These additions were conducted in a UV reactor (A=365 nm) and were catalyzed by DMPA and were not optimized. The monomers (11) are thus obtained in the form of viscous liquids.

In a previously dried Schlenk flask equipped with a magnet bar and a bubbler, 10 g of 10-undecen-1-ol (59 mmol) and 0.49 g of 2nd generation Grubbs catalyst (0.57 mmol) are dissolved in 50 mL of pentane, wherein the solvent was previously dried on CaH₂. The reaction medium is then stirred under nitrogen flow at room temperature. The progress of the metathesis is marked by the precipitation of the unsaturated diol, thus shifting the equilibrium of the reaction towards the formation of the products. Two days later, 2 mL of ethylvinyl ether is added to deactivate the Grubbs catalyst. The excess ethylvinyl ether is then removed by vacuum distillation. The product is then purified twice by recrystallization in cold pentane. The metathesized diol is obtained in the form of a white solid. The yield of the step is 44%.

The monomers 11 are then formed by reaction of thiol-ene with methyl thioglycolate. To this end, 4 g of the intermediate diol (13 mmol) and 4.1 g of methyl thioglycolate (39 mmol) are dissolved in 10 ml of dichloromethane. 0.34 g of DMPA (1.3 mmol) are added thereto. The reaction medium is then placed under UV irradiation in a dedicated reactor for this purpose (400 W, 315 nm≤λ≤400 nm). The progress of the reaction is monitored by 1H NMR spectroscopy until complete disappearance of the characteristic signals of the double bond. Excess methyl thioglycolate is then removed by vacuum distillation. The products are finally purified by flash chromatography on a silica column, on the basis of a cyclohexane/ethyl acetate elution (85/15:v/v). The monomers 11 are obtained in the form of viscous liquids. The yield of the step is 91%.

### Example 6: Preparation of Hyperbranched Architecture Polyesters from Monomers (7), (8) and (9)

The monomers (7), (8) and (9) were polymerized in bulk (in the molten state) in the presence of 1.5% by weight of catalyst (anhydrous zinc acetate or TBD) according to the following procedure:
Pre-drying during which the monomer is heated alone to 90° C. under dynamic vacuum for one hour in order to eliminate all traces of solvent
1st polymerization stage under nitrogen flow at 120° C. for 2 hours
2nd phase of dynamic vacuum polymerization at 160° C. for 13 hours

All the polymerizations were carried out in bulk (in the molten state) in a Schlenk tube equipped with a magnetic bar, according to the following procedure. A first pre-drying step consists in heating the monomer alone under dynamic vacuum to 90° C. above its melting point in order to eliminate all traces of solvent. One hour later, the temperature is set to T₁ (° C.). The reaction medium is placed under a stream of nitrogen and stirred for 2 hours in the presence of 1.5% m of catalyst, except for the cases mentioned. In fact, to control the polymerization of the monomers (7) and (11), lower concentrations were tested (0.75 to 1% m). After the 2 hours of oligomerization at T₁ (° C.), the temperature is set to T₂ (° C.) and the reaction medium is placed under dynamic vacuum until the viscosity suddenly increases. The conditions were optimized as indicated below. The monomers 7 were polymerized at T₁=120° C. and T₂=160° C. The presence of a second primary alcohol increases the reactivity of the monomers (9). A lower temperature of polymerization T₂=140° C. was therefore preferred. On the contrary, the presence of a single secondary alcohol forced us to move to much higher temperatures of T₁=180° C. and T₂=200° C. in order to polymerize the monomers (8). Finally, the monomers (10) and (11) were polymerized at T₁=30 to 90° C. (see Example 7 below). In each case, satisfactory conversions were achieved (>95%). The hyperbranched polyesters were obtained in the form of viscous colored liquids (yellow, orange to brown hues).

The following results were obtained:

| | | Time | Conv.^{a} | Mₙ^{a} | M_{w}^{a} | | T_{g}^{b} | T_{d}^{10%} c |
|---|---|---|---|---|---|---|---|---|
| Monomer | Catalyzer | (hours) | (%) | (g · mol−1) | (g · mol−1) | Ð^{a} | (° C.) | (° C.) |
| 7 | Zn(OAc)₂ | 8 | 99 | 10 040 | 41 870 | 4.17 | −36.5 | 319 |
| | TBD | 8 | 100 | 9 340 | 37 800 | 4.04 | −36 | 317 |
| 8 | Zn(OAc)₂ | 8 | 37 | 1 515 | 1 730 | 1.14 | n.d. | n.d. |
| | TBD | 8 | 41 | 2 060 | 2 515 | 1.22 | n.d. | n.d. |
| 9 | Zn(OAc)₂ | 4 | 86 | 2 500 | 4 640 | 1.86 | n.d. | n.d. |
| | TBD | 6 | 97 | 4 100 | 16 100 | 3.9 | n.d. | n.d. |
| | TBD^{d} | 8 | 95 | 4 680 | 31 950 | 6.83 | n.d. | n.d. |

^{a}SEC in THF (PS calibration),
^{b}DSC,
c TGA,
^{d}Temperature of the second polymerization phase lowered to 140° C.
T_{d}^{10%}: degradation temperature at 10%

The macromolecular characteristics of hyperbranched samples were measured by steric exclusion chromatography in THF using calibration from polystyrene standards.

It was also found that the presence of a single primary alcohol significantly improves the reactivity of the synthons (monomers). In fact, in the space of 8 hours (instead of the usual 15 hours for monomers with secondary alcohol functions), hyperbranched polyesters of molar masses twice as high are obtained for total conversions (≥99%).

The samples of the compounds obtained were characterized by 1H NMR spectroscopy in CDCl₃.

The data obtained confirm that the primary alcohol has a higher reactivity than the secondary alcohol whatever the catalytic system used.

The tests carried out with the monomer (9) led to the formation of insoluble gels in the space of 6 hours with anhydrous zinc acetate and 8 hours with TBD, respectively. These results show the presence of two primary alcohols further increases the reactivity of this synthon.

The thermomechanical properties of the hyperbranched polyesters obtained from the monomer (7) were studied. The values of the glass transition temperature (T_{g}) and of the 10% degradation are of the same order of magnitude as those obtained with the samples synthesized from a monomer with secondary alcohol functions.

It has thus been found that the monomers (7), (8) and (9) make it possible to obtain polymers having a hyperbranched architecture.

This series of experiments has demonstrated that the presence of simple primary alcohols confers a greater reactivity to these multifunctional monomers in polymerization. In particular, polyesters of high molar masses (Mn=5 to 10 kg·mol⁻¹) could be synthesized from the monomers (7) and (9).

Other polymerization tests were carried out from the monomer (7) by testing other catalysts than Zn(OAc)₂ and TBD.

Catalyst screening was extended to include many other metal systems (titanium, antimony and tin oxides) and organic systems, as well as a strong base, sodium methanolate.

The results are summarized in the table below.

| | Conv.^{a} | Mn^{a} | Mw^{a} | | |
|---|---|---|---|---|---|
| Catalyzer^{c} | (%) | (g · mol⁻¹) | (g · mol⁻¹) | Ð^{a} | OH(I)/OH(II)^{b} |
| Ti(OiPr)₄ | 74 | 1 590 | 1 880 | 1.18 | 45 |
| Sb₂O₃ | 34 | 1 230 | 1 285 | 1.04 | 17 |
| DBTO | 97 | 5 335 | 15 975 | 2.99 | 1.37 |
| Tin octanoate | 99 | 5 900 | 23 070 | 3.91 | 9.69 |
| m-TBD | 46 | 1 250 | 1 330 | 1.06 | n.d. |
| NaOMe | 100 | 7 110 | 29 985 | 4.22 | 5.18 |

^{a}SEC in THF (PS calibration),
^{b}1H NMR,
^{c}1.5% by weight
OH(I)/OH(II): ratio of reactivity of primary alcohols to secondary alcohols (this factor gives information on the branch density of the polymers obtained)

Procedure: Pre-drying, 2 hours at 120° C. under nitrogen flow, 6 hours at 160° C. under dynamic vacuum.

Of the metal catalysts, tin oxides catalyze the polymerization of synthon 7 in a particularly effective manner. Molar masses of the order of 5-6 kg·mol⁻¹ are reached for almost all the conversions (>97%). It is interesting to note that DBTO is particularly efficient for branched architecture.

The polymerization of the monomers (7) was also tested with titanium tetrabutanolate (Ti(OBᵤ)₄) as a catalyst according to the protocol below:

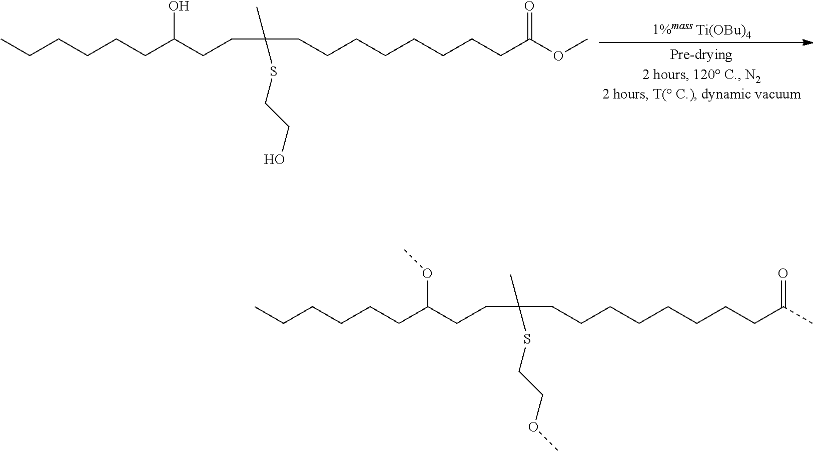

| | Conv.^{a} | Mn^{a} | Mw^{a} | | |
|---|---|---|---|---|---|
| T (° C.) | (%) | (g · mol⁻¹) | (g · mol⁻¹) | Ð^{a} | OH(I)/OH(II)^{b} |
| 120 | >98 | 5 860 | 21 960 | 3.75 | 9.88 |
| 140 | >98 | 9 890 | 113 555 | 11.5 | 8.78 |

^{a}SEC in THF (PS calibration),
^{b}1H NMR

By using a catalyst concentration at 1% m, compounds of molar masses similar to the previous results obtained (of the order of 6 to 10 kg·mol⁻¹) are formed in only 4 hours.

Whatever the polymerization temperature, the materials obtained by titanium tetrabutanolate catalysis have a rather linear architecture, wherein the reactivity of the primary alcohols is up to 10 times higher than that of the secondary alcohols.

Finally, the thermal properties of hyperbranched polyesters obtained from monomer 7 were studied. These materials exhibit a completely amorphous behavior with glass transition temperatures in the interval −41.7° C. to −34.2° C. and good thermal stability. The 10% degradation temperatures range from 311° C. to 328° C., which are ‘standard’ values for polymeric materials derived from oleaginous resources.

### Example 7: Preparation of Hyperbranched Architecture Polyesters from Monomer (9) with an Enzyme

A polymerization test was carried out by enzymatic catalysis. The protein in question is Candida Antartica lipase, commonly known as CaIB.

The monomer 9 was polymerized in bulk, melted at 60° C. under a flow of nitrogen in an open circuit (direct connection to a bubbler) in order to eliminate the methanol generated during the reaction and thus to shift the equilibrium towards the formation of the polymer. The lipase used is an enzyme supported on acrylic resin provided by Chiral Vision (Immozyme). It is inserted at 10% mass at the end of the pre-drying phase during which the monomer is heated to 90° C. under dynamic vacuum.

After one night only, the viscosity of the medium is such that agitation may no longer be ensured. The reaction medium is then solubilized in THF, and the insoluble enzyme filtered.

The conversion is satisfactory (95%) and the correct molar masses (Mn=3200 g·mol⁻¹, Ð=5.46).

This isolated test shows that an enzymatic catalysis is a solution adapted to the reactivity of this synthon.

### Example 8: Preparation of Hyperbranched Architecture Polyesters from Monomers (10) and (11)

The monomers (10) and (11) were found to be even more reactive than previous monomers.

The polymerization tests below were carried out with TBD as a catalyst.

The following results were obtained:

| | T₁ | T₂ | Time | Conv.^{a} | Mn^{a} | |
|---|---|---|---|---|---|---|
| Monomer | (° C.) | (° C.) | (hours) | (%) | (g · mol−1) | Ð^{a} |
| 11 | 30 | 30 | 4 | 91 | 3 950 | >11.3 |
| 10 | 90 | 90 | 3 | >99 | 9 950 | 5.01 |

^{a}SEC in the THF (PS calibration)

The hyperbranched polymers synthesized from the monomers 10 and 11 were found to be semi-crystalline:

| | Mn^{a} | | T_{g}^{b} | Tₘ^{b} | T_{c}^{b} | T_{d}^{5%} ^{c} |
|---|---|---|---|---|---|---|
| Monomer | (g · mol−1) | Ð^{a} | (° C.) | (° C.) | (° C.) | (° C.) |
| 11 | 3 950 | >11.3 | −40 | −20 | −35 | 266 |
| 10 | 5 850 | 2.79 | −56 | −29 | −44 | 298 |
| | 6 900 | 5.90 | −51 | −26 | −42 | 284 |

^{a}SEC in THF (PS calibration),
^{b}DSC,
^{c} TGA
Tₘ: melting temperature
T_{c}: crystallization temperature
T_{d}^{5%}: degradation temperature at 5%

Placed under the same conditions as previously described in Example 5, in the presence of 1.5% by weight of catalyst TBD, for the monomer (11), the reaction medium completely crosslinked during the first hour of oligomerization at 120° C. under nitrogen flow. Thus, an experiment was conducted by decreasing the temperature to 30° C. to obtain a fully soluble polymer sample.

The monomer (10) also made it possible to obtain hyperbranched polyesters with high molar masses (Mn=6-10 kg·mol⁻¹) for total conversions (>96%).

The hyperbranched polyesters obtained from compounds (10) and (11) were successfully characterized by 1H NMR spectroscopy.

This example reveals that the spacing of the reactive functions is also important. The multifunctional precursors (10) and (11) are even more reactive than the monomers (7), (8) and (9). They make it possible to generate hyperbranched polyesters of high molar masses.

1. Compound obtainable by a method comprising at least one polymerization step of at least one monomer of formula (I) below: 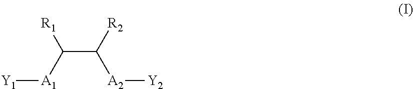
in which:
Y₁ is H, an OH group or a COORₐ group, wherein Rₐ represents H or a linear or branched alkyl group comprising from 1 to 6 carbon atoms,
Y₂ is an OH or COORₐ group, wherein Rₐ is as defined above, wherein it is understood that:
when Y₁ is H, then Y₂ is COORₐ,
when Y₁ is OH, then Y₂ is OH,
when Y₁ is COORₐ, then Y₂ is COORₐ,
wherein R₁ and R₂ are defined as follows:
either R₁ is H and R₂ is a group of formula —S-A₃-X,
or R₁ is a group of formula —S-A₃-X and R₂ is H,
wherein A₃ represents a linear or branched alkylene radical comprising from 1 to 12 carbon atoms, and
wherein X represents an OH or COOR_{b} group, wherein R_{b} is H or a linear or branched alkyl group comprising from 1 to 6 carbon atoms,
A₁ is a linear or branched alkylene radical comprising from 1 to 20 carbon atoms, optionally substituted by at least one OH group, wherein the alkylene radical is further substituted by a side chain —S-A₃-X, wherein A₃ and X are as defined above,
A₂ is a linear or branched alkylene radical comprising from 1 to 20 carbon atoms, optionally substituted by at least one OH group,
it being understood that at least one of the groups X, Y₁ and Y₂ is COORₐ or COOR_{b}, and wherein at least one of the groups A₁, A₁, X, Y₁ or Y₂ comprises an OH group,
wherein the total number of functions COORₐ, COOR_{b} and OH is at least 3, wherein the polymerization step is carried out in the presence of a catalyst selected from the group consisting of: Zn(OAc)₂, Ti(OBu)₄, Ti(OiPr)₄, Sb₂O₃, stannous octanoate, dibutyltin oxide, 7-methyl-1,5,7-triazabicyclo[4.4.0]dec-5-ene, NaOMe, 1,5,7-triazabicyclo[4.4.0]dec-5-ene and Lipase B Candida Antartica

2. Compound according to claim 1, wherein the monomer of formula (I) is a monomer of formula (II) below: 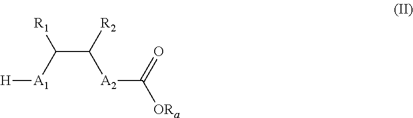
in which A₁, R₁, R₂, A₂ and Rₐ are as defined in claim 1.

3. Compound according to claim 1, wherein the monomer of formula (I) is a monomer of formula (II-1) below: 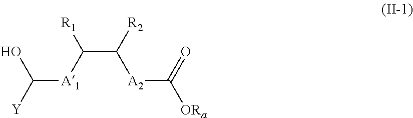
in which:
R₁, R₂, A₂ and Rₐ are as defined in claim 1,
A′₁ is a linear or branched alkylene radical comprising from 1 to 6 carbon atoms, and
Y is a linear or branched alkyl group comprising from 1 to 10 carbon atoms.

4. Compound according to claim 1, wherein X is OH in formula (I).

5. Compound according to claim 1, wherein X is COOR_{b} in formula (I), R_{b} being as defined in claim 1.

6. Compound according to claim 1, wherein the monomer of formula (I) is a monomer of formula (III) below: 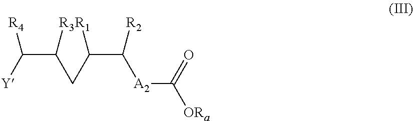
in which:
A₂ and Rₐ are as defined in claim 1,
R₁ and R₂ are defined as follows:
either R₁ is H and Rₐ is a group of formula —S-A₃-OH,
or R₁ is a group of the formula —S-A₃-OH and R₂ is H,
A₃ being as defined in claim 1,
R₃ and R₄ are defined as follows:
either R₃ is H and R₄ is a group of formula —S-A₃-OH,
or R₃ is a group of the formula —S-A₃-OH and R₄ is H,
A₃ being as defined in claim 1,
Y′ is a linear or branched alkyl group comprising from 1 to 10 carbon atoms.

7. Compound according to claim 1, wherein the monomer of formula (I) is a monomer of formula (IV) below: 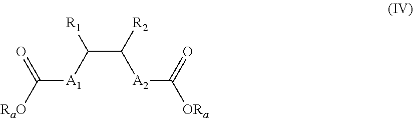
in which:
A₁, A₂ and Rₐ are as defined in claim 1, and
R₁, and R₂ are defined as follows:
either R₁ is H and R₂ is a group of formula —S-A₃-OH,
or R₁ is a group of the formula —S-A₃-OH and R₂ is H,
A₃ being as defined in claim 1.

8. Compound according to claim 1, wherein the monomer of formula (I) is a monomer of formula (V) below: 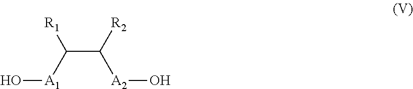
in which:
A₁ and A₂ are as defined in claim 1, and
R₁ and Rₐ are defined as follows:
either R₁ is H and Rₐ is a group of formula —S-A₃-COOR_{b},
or R₁ is a group of formula —S-A₃-COOR_{b} and Rₐ is H,
A₃ and R_{b} being as defined in claim 1.

9. Compound according to claim 1, wherein the monomer of formula (I) corresponds to one of the following formulas:

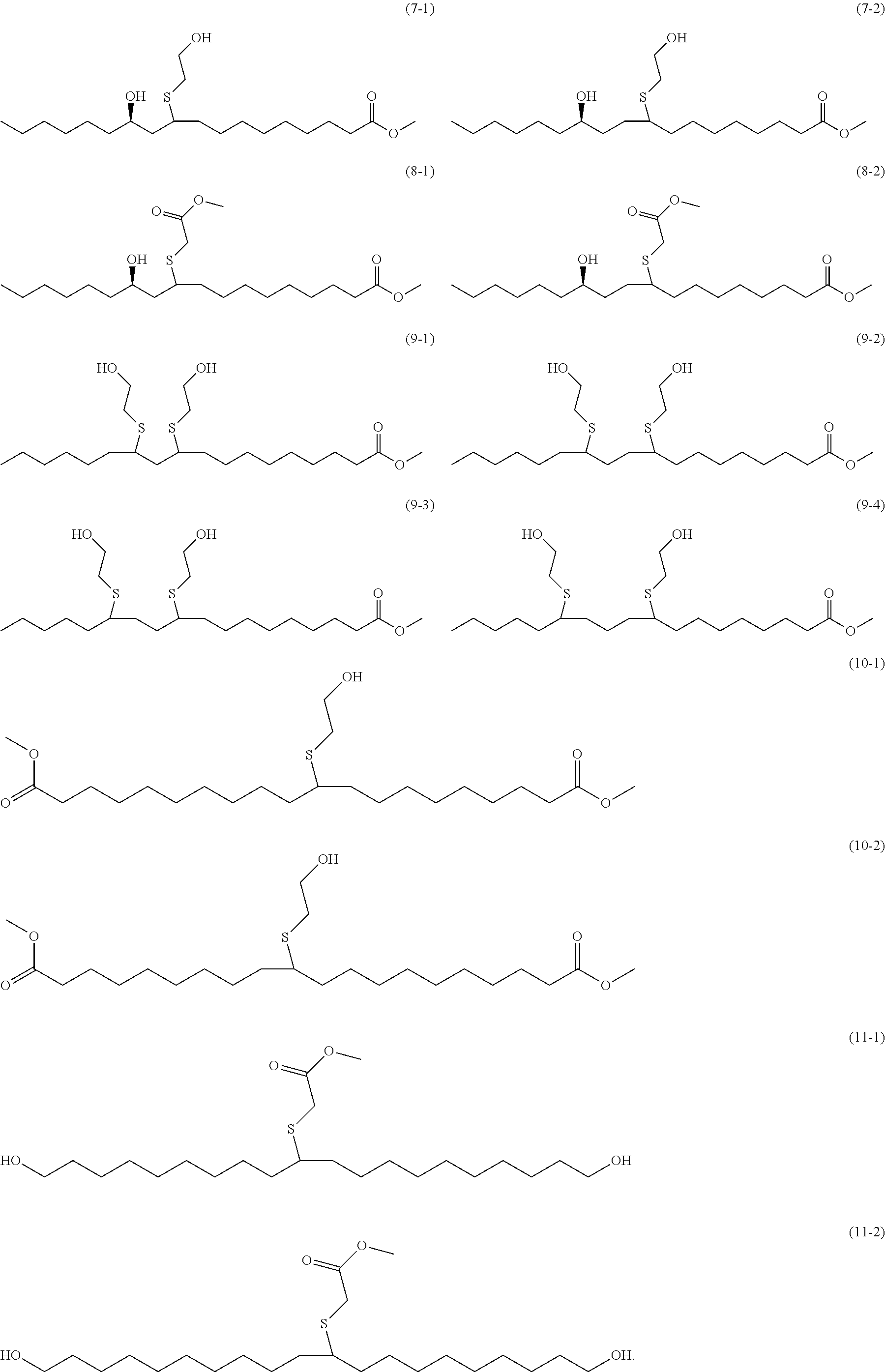

10. Method for the preparation of a compound of claim 1, comprising at least one polymerization step of a monomer of formula (I) as defined in claim 1, in the presence of a catalyst selected from the group consisting of: Zn(OAc)₂, Ti(OBu)₄, Ti(OiPr)₄, Sb₂O₃, stannous octanoate, dibutyltin oxide, 7-methyl-1,5,7-triazabicyclo[4.4.0]dec-5-ene, NaOMe, 1,5,7-triazabicyclo[4.4.0]dec-5-ene and Lipase B Candida Antartica.

11. Method according to claim 10, wherein the catalyst is Zn(OAc)₂, 1,5,7-triazabicyclo[4.4.0]dec-5-ene or NaOMe.

12. Method according to claim 10, wherein the catalyst content ranges from 0.05% to 20% by weight relative to the total weight of monomer of formula (I).

13. Method of claim 10, in which the polymerization is carried out (a) by heating the monomer of formula (I) of claim 1 in the presence of the catalyst, to a temperature T₁ from 30° C. to 130° C., for a period of from 1 hour to 48 hours, then optionally (b) under dynamic vacuum at the temperature T₁ for a period of 1 hour to 48 hours, and optionally (c) by additional heating to a temperature T₂ of 90° C. to 180° C., for a period of 1 hour to 48 hours.

14. Monomer of formula (I): 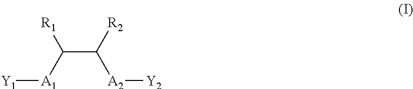
in which Y₁, Y₂, A₁, A₂, R₁ and R₂ are defined claim 1.

15. Monomer according to claim 14, having one of the following formulas (7-1), (7-2), (8-1), (8-2), (9-1), (9-2), (9-3), (9-4), (10-1), (10-2), (11-1) and (11-2): [chemical structure]
